(19) United States
(12) Patent Application Publication
(10) Pub. No.: US 2007/0073279 A1
(43) Pub. Date: Mar. 29, 2007

(54) Variable continuous wave laser

(75) Inventors: T. Scott Rowe, Dana Point, CA (US); Christopher Horvath, Irvine, CA (US); Bryan Somen, Santa Ana, CA (US); Bruno X. Lassalas, Irvine, CA (US); Stanley C. Polski, Fullerton, CA (US)

Correspondence Address: ALCON, IP LEGAL, TB4-8, 6201 SOUTH FREEWAY, FORT WORTH, TX, 76134, US

(73) Assignee: Alcon, Inc.

(21) Appl. No.: 11/541,111

(22) Filed: Sep. 29, 2006

Related U.S. Application Data
(60) Provisional application No. 60/721,648, filed on Sep. 29, 2005.

(51) Int. Cl. A61B 18/18; A61F 9/008

(52) U.S. Cl. 606/11; 606/13; 606/5

(57) ABSTRACT

A continuous wave laser is provided to produce isolated surgical effects within selected tissue layers. The continuous wave laser includes a laser source, an optical modulation device, and a system controller. The laser source produces a laser beam which is provided to the optical modulation device. The optical modulation device modulates the laser beam in order achieve isolated surgical effects within selected tissue layers. The system controller drives the laser source and the optical modulation device to achieve the isolated surgical effects. The system controller may direct the laser beam delivered to the selected tissues comprise a series of modulated bursts which further comprise modulated micro bursts. These bursts and micro bursts may be modulated in amplitude, duration and separation.

### CROSS-REFERENCE TO RELATED APPLICATIONS

This application claims priority under 35 U.S.C. §119 to U.S. Provisional Patent Application No. 60/721,648, filed Sep. 29, 2005, the entire contents of which are incorporated herein by reference.

### TECHNICAL FIELD OF THE INVENTION

The present invention relates generally to laser sources for surgical procedures and, more particularly, to a surgical laser and method of using the laser in continuous wave mode while varying various parameters of the delivered laser beam.

### BACKGROUND OF THE INVENTION

A number of ophthalmic surgical procedures performed on a patient's eye, such as on the retina, require illuminating a select portion of the eye with a light spot, typically provided by a laser, having a desired size. In one such procedure, commonly known as “photo-dynamic therapy”, an agent, which is harmless in the absence of light activation, is initially administered intravenously to the patient. Subsequently, abnormally highly-vascularized retinal tissue containing the agent is illuminated with laser light having a selected wavelength to activate the agent. The activated agent can destroy the abnormal tissue or have other therapeutic affect.

In another ophthalmic surgical procedure, typically referred to as retinal coagulation, a laser light spot is directed to a selected portion of a patient's retina to deposit energy, thereby causing coagulation of the local tissue. Such a photocoagulation procedure can be employed, for example, to seal leaky blood vessels, destroy abnormal blood vessels, or seal retinal tears.

Other ophthalmic and non-ophthalmic surgical procedures are also known to utilize lasers for various purposes. For example, in a LASIK procedure an excimer laser is used to photo-ablate corneal tissue to shape a cornea and correct refractive errors. Other examples of surgical procedures utilizing lasers include laser sclerlostomy, trabeculectomy, and general endoscopic microsurgical applications, including neural, arthroscopic, and spinal chord surgery. These and other medical procedures may derive great benefit from a continuous wave, variable output laser.

Laser systems have been widely used in the medical field to treat tissue in these procedures and others. The high-intensity energy of a laser beam can be concentrated into a small cross sectional area and used to treat different types of tissues to accomplish different functions, such as cutting, cauterizing, cell destruction, etc. Each type of tissue generally reacts positively to radiation of a specific wavelength. Therefore, laser systems operating at various fundamental wavelengths are advantageous for different types of operations. For example, in ophthalmic surgical operations, it has been found that a YAG type laser generating a wavelength of 1064 or 1320 nanometers (nm) is especially advantageous for cyclophotocoagulation or capsulotomies. Radiation wavelengths in the yellow range of the visible spectrum have been found to be advantageous in the treatment of retinal telangiectatic or intra-retinal vascular abnormalities. Radiation wavelengths in the orange range of the visible spectrum have been found to be advantageous in the treatment of parafoveolar subretinal neovascularization in hypopigmented individuals. Radiation wavelengths in the red range of the visible spectrum have been found to be advantageous in the treatment of foveolar subretinal neovascularization, intraocular tumors such as choroidal malignant melanomas and retinoblastomas, as well as in the production of panretinal photocoagulation. Radial wavelengths in the blue/green range of the visible spectrum have been found to be excellent photocoagulators.

Lasers producing different radiation wavelengths can thus be used to treat different physical diseases. While most lasers are not monochromatic and produce radiation with a variety of wavelengths, the radiation spectrum of most lasers is relatively narrow with radiation output peaks occurring at fairly well defined wavelength lines. These radiation output peaks can affect different tissues to varying extents.

In particular, treatment of retinal tissue by irradiating retinal tissue with laser light has demonstrated that different tissue layers absorb laser energy and heat up at different rates depending on many different parameters, such as: absorption coefficient for a specific wavelength, scattering coefficient, thickness of individual layers, momentary temperature of the individual layers, thermal conductivity of the individual layers and their thermal realization times, and power and exposure time of the laser. The retina is known to have layers with significant variations in absorption, scattering, thickness and other parameters.

Typically, the outermost tissue layers, or those layers having the highest absorption coefficient, experience the highest heating effect from an impinging continuous wave laser beam, since they are exposed to the highest laser power or absorb a greater percentage of the deposited laser power. However, there exist certain surgical conditions in which it would be preferable to target a deeper-lying tissue layer for maximum heat retention (coagulation). In these instances, it would be preferable to isolate the heating effect to a selected, possibly deeper, layer as much as possible. This could be desirable in order to isolate the surgical effects of a laser from certain retinal layers and thereby minimize collateral damage to these surrounding layers. Currently existing laser sources do not provide the capability to vary the pulse rate, power rate, cycle rate or combinations of these parameters to localize and select the tissue to be affected by an incident laser beam and thus minimize collateral damage to surrounding tissue.

### BRIEF DESCRIPTION OF THE DRAWINGS

For a more complete understanding of the present invention and the advantages thereof, reference is now made to the following description taken in conjunction with the accompanying drawings in which like reference numerals indicate like features and wherein:

FIG. 1 provides an overview of a laser surgical procedure where a laser beam is used to remove optical tissue;

FIG. 2 schematically illustrates the interaction of an incident laser beam pulse within optical tissues;

FIG. 3 provides a schematic diagram of one possible pattern of micro-pulse laser beam bursts from an embodiment of the variable continuous-wave laser of the present invention operating in continuous-wave mode, but having its output varied by use of a optical modulation device;

FIG. 4 is a schematic illustration of an exemplary non-symmetrical custom wave form that may be a wave form best-suited for localizing incident laser beam effects for a particular tissue layer when using an embodiment of the present invention;

FIG. 5 provides a functional diagram of a basic variable continuous wave laser setup in accordance with an embodiment of the present invention; and

FIG. 6 is a logic flow diagram in accordance with an embodiment of the present invention.

### SUMMARY OF THE INVENTION

The present invention provides a variable continuous wave laser that substantially eliminates or reduces disadvantages and problems associated with previously developed systems and methods. More specifically, addresses the need for a laser system that can provide the capability to vary power, pulse duration and duty cycle from pulse-to-pulse and/or within a burst of micro-pulses output from the laser system so as to optimize the localization of laser beam thermal effects and better protect surrounding tissues layers from heating by the laser beam.

### DESCRIPTION OF THE INVENTION

Preferred embodiments of the present invention are illustrated in the figures, like numerals being used to refer to like and corresponding parts of the various drawings.

Embodiments of the present invention substantially address the above identified needs as well as others. One embodiment of the variable continuous-wave laser of the present invention comprises a laser source capable of providing flexibility in pulse duration and on-the-fly power changes necessary to isolate the surgical effects of the laser beam produced by the laser source to selected tissue layers, such as selected retinal tissue layers, and thereby minimize collateral damage to neighboring tissue layers. A method using such a laser will overcome the prior art problems associated with modulating laser cavity power, which is very difficult to do in a controlled fashion on such a time scale.

The application of lasers to vision correction has opened new possibilities for treating nearsightedness, farsightedness, astigmatism, and other conditions of the eye. Specifically, Laser technology has allowed the development of modem laser techniques that are collectively known as laser vision correction.

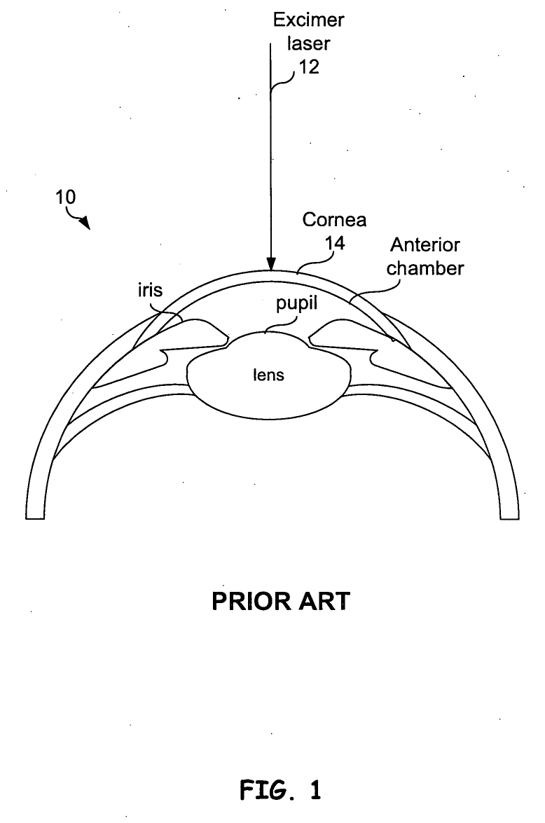

These laser vision correction techniques apply laser energy to selected tissues of eye 10 as shown in FIG. 1. For example a laser vision correction technique may employ a cool beam of light (such as Excimer laser beam 12) to remove microscopic amounts of tissue. The removal of this tissue changes the shape of cornea 14 in order to allow sharper focusing of images and reducing a patient's dependence on glasses and/or contact lenses. Laser vision corrective surgeries include but are not limited to laser-assisted in situ keratomileusis (LASIK), laser epithelial keratomileusis (LASEK), epi-LASIK, automated lamellar keratoplasty (ALK), photo ablation procedures such as photo refractive keratectomy (PRK), and other like procedures.

In these procedures, the quality of the results of the laser vision correction may depend upon the ability of the laser 12 to precisely deliver laser energy to selected tissues within the eye 10. Accurately removing tissue with laser 12, in turn may at least in part depend on the ability to accurately align and control the laser.

The embodiments of the present invention provide the ability to change laser power, pulse duration and laser “off time” on-the-fly within a laser burst to maximize a localization effect. Typically, the outermost tissue layers, or those layers having the highest absorption coefficient, experience the highest heating effect from an incident continuous-wave laser beam, since they are exposed to the highest laser power or absorb a greater percentage of the deposited laser power. The embodiments of the present invention provide a method and system directed to isolating the heating effect from an incident laser beam to a targeted, perhaps deeper layer. The embodiments of the present invention achieve these results by, for example, providing a burst of short laser pulses (e.g., 1 μs to 500 μs in pulse duration), which results in the absorption and scattering of the laser pulse interacting more with the tissue layers having a thermal relaxation roughly on the same time scale as the laser pulses. As the local temperatures of the different layers rise, some of the tissue properties, such as scattering, absorption and thermal conductivity, might change.

This change can be further useful to target a specific tissue layer by increasing or decreasing the pulse power of the incident laser beam in a continuous way to match the changes of the desired target tissue layer. The detailed interactions between the incident laser beam and the tissue layers are very complicated and linked by the changes of many parameters. Advanced computer simulations, as will be known to those familiar with the art, can be performed to predict the incident laser beam pulse/power configurations that are best suited to isolate heating of different layers. One particular layer of interest is the RPE layer. By targeting specific tissue layers, the collateral damage to neighboring tissue layers associated with certain surgeries and the prior art can be dramatically reduced and an improved surgical outcome can be achieved.

To enable the methodology of the embodiments of this invention, a laser source is needed which can create bursts of laser pulses in the 10 μs to 500 μs duration range while also capable of changing laser power “on-the-fly”, and ideally from pulse-to-pulse. The embodiments of the present invention provide such a laser source. Currently available prior-art lasers (at least those in the 532 nm range) cannot achieve this type of variable continuous-wave operation. The embodiments of the variable continuous-wave laser of the present invention can provide this functionality and can do so by providing a laser source that can operate in continuous-wave mode at the maximum required power for a desired surgery. An external (outside the laser cavity) Pockel-cell, with a driver of, for example, around 1 μs rise and fall time, can be used to modulate the laser beam output from the laser source to any desired shape and power amplitude. New, highly nonlinear RTP Pockel-cell crystals are of particular interest for incorporation into the present invention because they can be operated with a relatively low voltage signal of less than 1,000V, versus the typical required voltage of around 6,000V-8,000V. Such a Pockel-cell crystal can dramatically simplify the driver and the implementation of such a crystal and reduce its cost.

By varying the power setting, the pulse duration and the duty cycle from pulse-to-pulse, and, in a particular embodiment, from pulse-to-pulse within a burst of micro-pulses, the embodiments of the variable continuous-wave laser of the present invention can provide the ability to optimize the localization effect of thermal coagulation resulting from tissue absorption of the energy of an incident laser beam and therefore better protect surrounding tissue layers from damage.

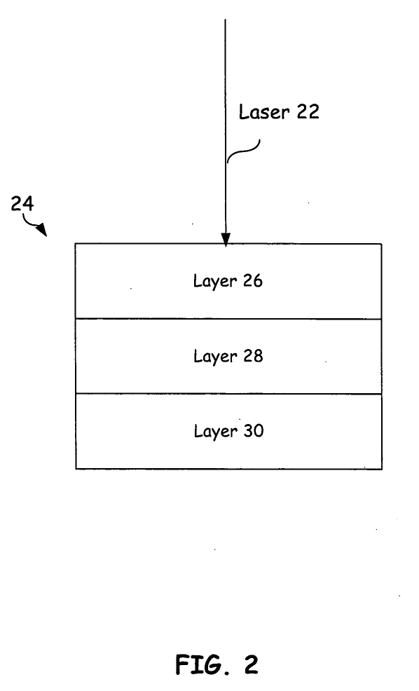

FIG. 2 schematically illustrates a very simplified stationary example of the interaction of an incident laser beam 22 pulse into tissue 24 without taking into account any scattering effects on the light beam or dynamic parameter changes. For a continuous-wave operated laser, layer 28 will absorb most of the incident laser beam energy and will heat up the most. In this example, tissue layer 28 has high absorption and fast relaxation. For a pulse laser mode of operation, during the laser “on” time, layer 28 will have the greater increase in temperature, but during the laser “off” time, it will also cool down much faster than layer 30. Layer 28 will eventually reach an equilibrium temperature where the heating amount during the “on” time and the cooling amount during the “off” time are the same. Depending on the exact timing of the laser pulses, it is possible to get layer 30 hotter than layer 28 because layer 30's thermal relaxation time is much longer (in this example) than the pulse period (as compared to the thermal relaxation time of layer 28). Even though the incident laser pulses have a smaller individual affect on layer 30 (low absorption) compared to layer 28 (high absorption), layer 30 will receive a greater cumulative effect because of the reduced cooling (slow relaxation) of layer 30 during the laser “off” time. The equilibrium temperature for layer 30 can thus be higher than that of layer 28, with the incident laser beam pulses having a greater effect on layer 30 than on layer 28.

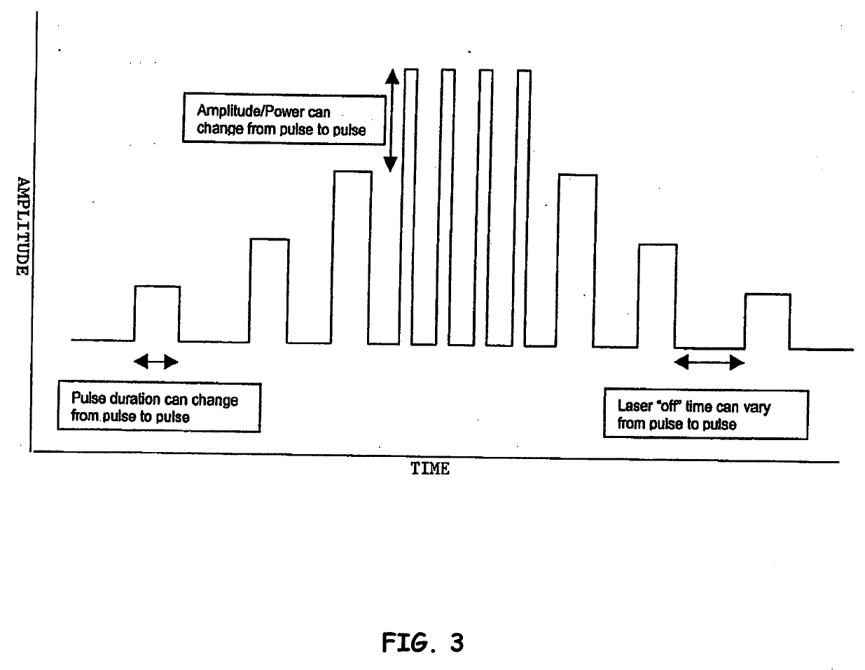

FIG. 3 provides a schematic diagram of one possible pattern of micro-pulse laser beam bursts from an embodiment of the variable continuous-wave laser of the present invention operating in continuous-wave mode, but having its output varied by use of a Pockel-cell. As can be seen from FIG. 3, the pulse duration, amplitude/power, and/or the laser “off” time can be varied from pulse-to-pulse (or any combination thereof). The example shown in FIG. 3 is exemplary only to illustrate the various parameters that can be changed to vary the output of an embodiment of the laser of the present invention. The pulse burst shape can be modeled and trialed experimentally to determine the best possible shape for targeting and localizing the laser effects to different tissue types and tissue layers. The embodiments of the present invention can take advantage of laser beam amplitude, pulse duration and “off” time variations to optimize the desired localization effect on a desired tissue or tissue layer once a tissue or tissue layer's properties are known.

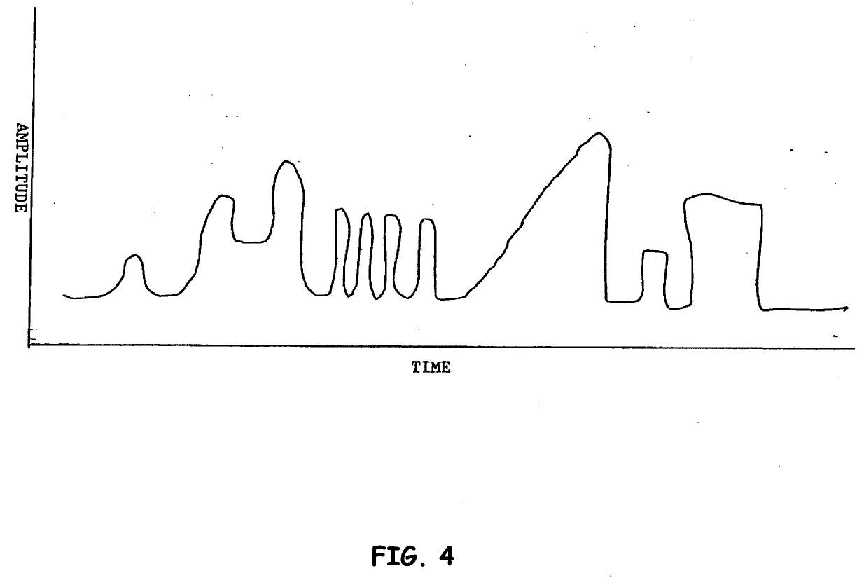

FIG. 4 is a schematic illustration of an exemplary non-symmetrical custom wave form that may be a wave form best-suited for localizing incident laser beam effects for a particular tissue layer when using an embodiment of the present invention. Such a wave form is for example purposes only to illustrate that such a wave form may be determined to be, from experimentation and known tissue properties, to have a desired effect on selected tissues in accordance with the teachings of this invention and that such a wave form is contemplated to be within the scope of this invention.

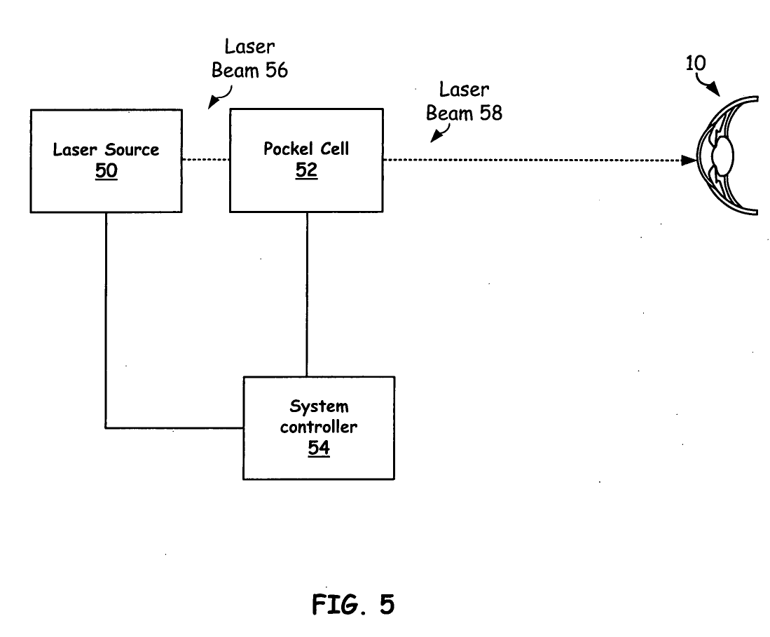

FIG. 5 provides a functional diagram of a basic variable continuous wave laser setup in accordance with an embodiment of the present invention. This optical setup includes laser source 50, Pockel Cell 52, and system controller 54. Laser source 50 produces a laser beam 56 which is supplied to the Pockel Cell 52. System controller 54 provides commands to the laser source 50 and Pockel Cell 52.

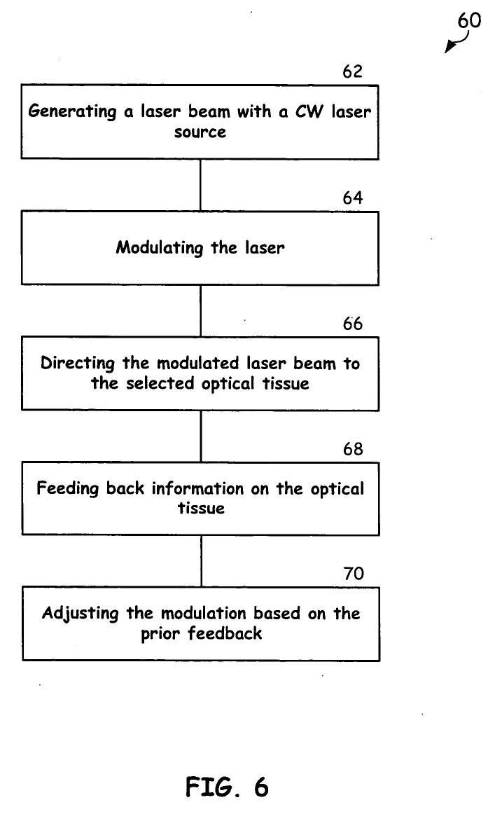

The system controller may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions stored in memory. The memory may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the system controller implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The memory stores, and the system controller executes, operational instructions corresponding to at least some of the steps and/or functions illustrated in FIG. 6.

FIG. 6 illustrates a method to deliver laser energy to selected optical tissues in accordance with embodiments of the present invention. Operations 60 begin with Steps 62 where a laser beam is generated with a continuous waive laser source. In Step 64 the generated laser beam is modulated. This laser beam may be made up of a series of bursts that further comprise a number of smaller bursts. Modulating the laser beam may involve modulating both the bursts and the smaller bursts in amplitude, separation, pulse length, phase, and frequency. This modulation may be done using an optical modulation device such as a Pockel cell. In Step 66, the modulated laser beam may be directed to selected optical tissues. As the modulated laser beam heats the selected optical tissue and adjacent optical tissues this information may be fed back to a system controller in Step 68. The system controller in Step 70 may adjust the modulation based on the prior feedback. This allows the modulation to be adjusted to achieve the desired isolated surgical effects.

In summary, A continuous wave laser is provided to produce isolated surgical effects within selected tissue layers. The continuous wave laser includes a laser source, an optical modulation device, and a system controller. The laser source produces a laser beam which is provided to the optical modulation device. The optical modulation device modulates the laser beam in order achieve isolated surgical effects within selected tissue layers. The system controller drives the laser source and the optical modulation device to achieve the isolated surgical effects. The system controller may direct the laser beam delivered to the selected tissues comprise a series of modulated bursts which further comprise modulated micro bursts. These bursts and micro bursts may be modulated in amplitude, duration and separation.

Embodiments of the present invention have the advantage that they provide an accurate and repeatable alignment mechanism that uses an actual expert laser path to perform measurements. The time associated with a manual geometry adjust high calibration is reduced or eliminated between patients and may be also performed between eyes of a bilateral case without any additional time penalty.

Additionally, the embodiments of the present invention may be used to automatically compensate for system misalignments from a variety of sources without requiring external mechanisms. Other aspects of the present invention may help maintain a stable operating temperature within the beam scanning mechanism in order to further reduce fluctuations in system performance.

Although the present invention is described in detail, it should be understood that various changes, substitutions and alterations can be made hereto without departing from the spirit and scope of the invention as described.

What is claimed is:

1. A continuous-wave laser operable to produce isolated surgical effects within selected tissue layers, comprising:
a laser source operable to produce a laser beam having a variable pulse duration and variable pulse power; and
a Pockel-cell operable to modulate the laser beam produced by the laser source wherein modulation of the laser beam provides isolated surgical effects within selected tissue layers; and
a system controller operable to drive the laser source and Pockel cell to selectively deposit laser energy within the selected tissue layer.

2. The continuous-wave laser of claim 1, wherein the pulse duration is based on knowledge of a thermal relaxation of the selected tissue layer.

3. The continuous-wave laser of claim 1, wherein the pulse duration is between about 1 μs and about 500 μs.

4. The continuous-wave laser of claim 1, wherein the laser beam delivered to the selected tissue layers comprises a burst of laser pulses.

5. The continuous-wave laser of claim 1, further comprising a feedback loop, wherein the system controller receives information on tissue layers exposed to the laser beam.

6. The continuous-wave laser of claim 5, wherein the system controller directs the laser source and Pockel Cell to vary the pulse duration and pulse power based on the selected tissue and the information on tissue layers exposed to the laser beam.

7. The continuous-wave laser of claim 1, wherein the laser beam delivered to the selected tissue layers comprises modulated bursts of modulated micro laser pulses.

8. A continuous-wave laser operable to produce isolated surgical effects within selected tissue layers, comprising:
a continuous wave laser source operable to produce a laser beam; and
a Pockel-cell operable to modulate the laser beam produced by the laser source, wherein modulation of the laser beam provides isolated surgical effects within selected tissue layers, wherein the modulated laser beam comprises bursts of smaller bursts; and
a system controller operable to drive the laser source and Pockel cell to selectively deposit laser energy within the selected tissue layer.

9. The continuous-wave laser of claim 8, wherein the bursts are modulated in amplitude, separation and pulse length.

10. The continuous-wave laser of claim 8, wherein the smaller bursts are modulated in amplitude, separation and pulse length.

11. The continuous-wave laser of claim 8, wherein the bursts and smaller bursts are modulated based on knowledge of a thermal relaxation of the selected tissue layer.

12. The continuous-wave laser of claim 8, wherein the burst is between about 1 μs and about 500 μs.

13. The continuous-wave laser of claim 8, wherein Pockel cell comprises a nonlinear RTP Pockel Cell.

14. The continuous-wave laser of claim 8, further comprising a feedback loop, wherein the system controller receives information on tissue layers exposed to the laser beam.

15. The continuous-wave laser of claim 14, wherein the system controller directs the laser source and Pockel Cell to vary the pulse duration and pulse power based on the selected tissue and the information on tissue layers exposed to the laser beam.

16. A method to deliver laser energy to selected optical tissues comprising:
generating a laser beam with a continuous wave laser source;
modulating the laser beam wherein the modulated laser beam comprises a number of bursts and wherein the bursts further comprise a number of smaller bursts; and
directing the modulated laser beam to the selected optical tissue.

17. The method of claim 16, wherein modulating further comprises:
modulating the bursts in amplitude, separation and pulse length; and/or
modulating the smaller bursts in amplitude, separation and pulse length.

18. The method of claim 17, wherein modulating the laser beam is based on a knowledge of a thermal relaxation of the selected tissue layer.

19. The method of claim 17, wherein the burst is between about 1 μs and about 500 μs.

20. The method of claim 17, wherein modulating the laser beam is achieved with a Pockel cell.

21. The method of claim 17, further comprising feeding back information on tissue layers exposed to the laser beam to a system controller; and modulating the laser beam based on that information.
